(19) United States
(12) Patent Application Publication
(10) Pub. No.: US 2023/0405282 A1
(43) Pub. Date: Dec. 21, 2023

(54) CATHETER ASSEMBLY

(71) Applicant: TERUMO KABUSHIKI KAISHA, Tokyo (JP)

(72) Inventor: Shinichi Mizuno, Nakakoma-gun (JP)

(73) Assignee: TERUMO KABUSHIKI KAISHA, Tokyo (JP)

(21) Appl. No.: 18/455,747

(22) Filed: Aug. 25, 2023

Related U.S. Application Data
(63) Continuation of application No. PCT/JP22/09862, filed on Mar. 8, 2022.

(30) Foreign Application Priority Data
Mar. 17, 2021 (JP) 2021-043023

(51) Int. Cl. A61M 25/06; A61M 25/01; A61M 25/00

(52) U.S. Cl. CPC A61M 25/0625; A61M 25/0631; A61M 2025/0175; A61M 25/0102; A61M 25/0032; A61M 25/0643

(57) ABSTRACT

A catheter assembly includes: a catheter having a first lumen; a catheter hub joined to a proximal end of the catheter; a reinforcement needle inserted through the first lumen of the catheter, having a second lumen, and including a distal portion that is blunt; a reinforcement needle hub joined to a proximal end of the reinforcement needle; an inner needle inserted through the second lumen and including a sharp needle tip at a distal end thereof; an inner needle hub joined to a proximal end of the inner needle; a first fixing portion configured to fix the catheter hub and the reinforcement needle hub so as not to be detachable by an operation of pulling the catheter hub and the reinforcement needle hub in an axial direction; and a second fixing portion configured to fix the reinforcement needle hub and the inner needle hub to each other.

### CROSS-REFERENCE TO RELATED APPLICATIONS

This is a bypass continuation of PCT Application No. PCT/JP2022/009862, filed on Mar. 8, 2022, which claims priority to Japanese Application No. JP2021-043023, filed on Mar. 17, 2021. The entire contents of these applications are incorporated herein by reference.

### BACKGROUND

The present disclosure relates to a catheter assembly.

In the related art, a catheter assembly is used for continuous infusion of fluid into a blood vessel of a patient, or the like. A catheter assembly includes a relatively flexible catheter (outer needle) and an inner needle inserted into a lumen of the catheter and including a sharp needle tip at a distal end thereof (for example, JP2008-173463A).

In a procedure of indwelling such a catheter assembly in a blood vessel of a patient, first, a user such as a doctor or a nurse punctures the catheter assembly into skin of the patient in a state in which the needle tip of the inner needle protrudes from a distal end of the catheter. When a blade surface of the needle tip of the inner needle enters the blood vessel, blood flows back (also referred to as flashback) through the inner needle, and the user visually observes securing the blood vessel. Thereafter, the inner needle is pulled out and the catheter is advanced into the blood vessel, thus indwelling the catheter in the blood vessel.

In the catheter assembly, because the catheter is flexible, it is less likely to damage a posterior wall of the blood vessel, but it is difficult to transmit an operation force only by the catheter and to push forward the catheter into the blood vessel. Therefore, the user may perform an operation of advancing the catheter while leaving the inner needle having high rigidity inside the catheter in order to facilitate the advancing of the catheter.

However, although the user operates the inner needle with intention of not allowing a blade edge of the inner needle to protrude from the distal end of the catheter, the blade edge of the inner needle may actually protrude from the catheter and may unintentionally damage the posterior wall of the blood vessel.

### SUMMARY

Therefore, an object of one embodiment is to provide a catheter assembly in which a catheter can be easily advanced without damaging a posterior wall of a blood vessel.

According to one aspect of the disclosure, a catheter assembly includes: a catheter having a first lumen; a catheter hub joined to a proximal end of the catheter; a reinforcement needle inserted through the first lumen of the catheter, having a second lumen, and including a distal portion formed blunt; a reinforcement needle hub joined to a proximal end of the reinforcement needle; an inner needle inserted through the second lumen of the reinforcement needle and including a sharp needle tip at a distal end thereof; an inner needle hub joined to a proximal end of the inner needle; a first fixing portion configured to fix the catheter hub and the reinforcement needle hub so as not to be detachable by an operation of pulling the catheter hub and the reinforcement needle hub in an axial direction; and a second fixing portion configured to fix the reinforcement needle hub and the inner needle hub to each other. The reinforcement needle has higher rigidity than the catheter and protrudes from a distal end of the catheter, and the inner needle protrudes from a distal end of the reinforcement needle.

According to certain embodiments of the catheter assembly of the present disclosure, the catheter can be easily advanced without damaging a posterior wall of a blood vessel.

### BRIEF DESCRIPTION OF THE DRAWINGS

FIG. 1 is a plan view of a catheter assembly according to a first embodiment;

FIG. 2 is a cross-sectional view of a vicinity of a distal end of the catheter assembly in FIG. 1;

FIG. 3 is a cross-sectional view of a vicinity of a proximal portion of the catheter assembly in FIG. 1;

FIG. 4 is a plan view of a vicinity of a distal end of a reinforcement needle in FIG. 1;

FIG. 5A is a cross-sectional view of a proximal portion of a catheter hub in FIG. 1, and FIG. 5B is a cross-sectional view of a reinforcement needle hub in FIG. 1 as viewed from a distal end toward a proximal end thereof;

FIG. 6A is a view illustrating a step of puncturing a blood vessel with the catheter assembly in FIG. 1, and FIG. 6B is a view illustrating a step of pulling out an inner needle from the catheter assembly that is punctured into the blood vessel;

FIG. 7A is a view illustrating a step of advancing a catheter and the reinforcement needle inside the blood vessel, and FIG. 7B is a view illustrating deformation of the catheter and the reinforcement needle;

FIG. 8A is a plan view of a vicinity of a distal end of a reinforcement needle according to a second embodiment, and FIG. 8B is a cross-sectional view of the reinforcement needle taken along a line VIIIB-VIIIB in FIG. 8A;

FIG. 9 is a plan view of a reinforcement needle according to a third embodiment;

FIG. 10A is a plan view of a reinforcement needle according to a fourth embodiment, and FIG. 10B is a cross-sectional view of the reinforcement needle taken along a line XB-XB in FIG. 10A;

FIG. 11 is a plan view of a catheter assembly according to a fifth embodiment;

FIG. 12A is a cross-sectional view of a latch section in FIG. 11 in a fixed state, FIG. 12B is a cross-sectional view of the latch section in FIG. 11 in a released state, and FIG. 12C is a cross-sectional view of the latch section after a lock pin is removed; and

FIG. 13 is a plan view of a catheter assembly according to a sixth embodiment.

### DETAILED DESCRIPTION

Hereinafter, preferred embodiments of a catheter assembly will be described in detail with reference to the accompanying drawings.

### First Embodiment

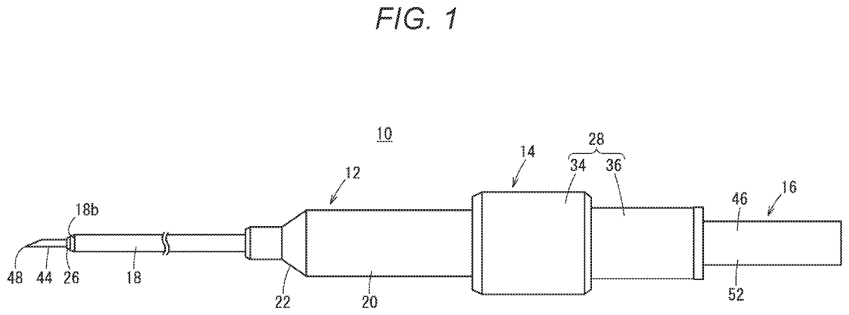

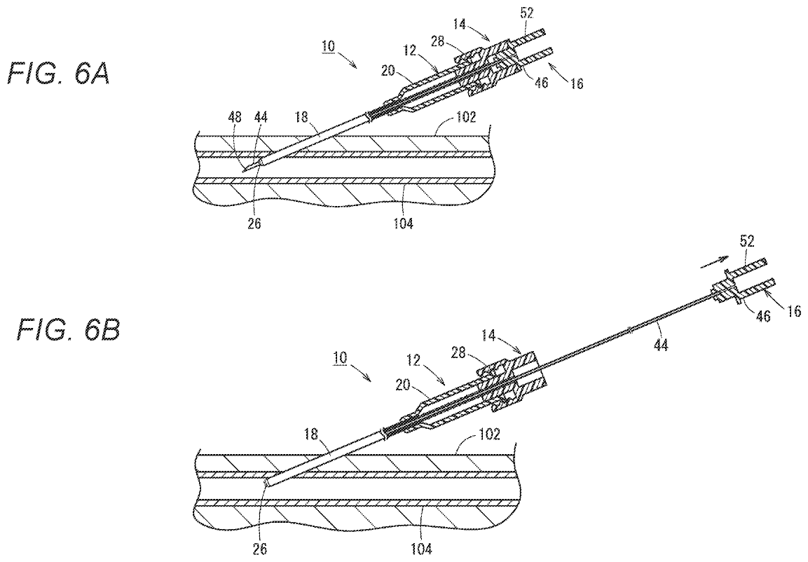

As illustrated in FIG. 1, a catheter assembly 10 according to the present embodiment includes a catheter unit 12, a reinforcement needle unit 14 inserted inside the catheter unit 12, and an inner needle unit 16 inserted inside the reinforcement needle unit 14. The catheter assembly 10 is used for administration of infusion fluid or the like by indwelling the catheter unit 12 in a blood vessel 104 (see FIG. 6A). The reinforcement needle unit 14 and the inner needle unit 16 are used when the catheter unit 12 punctures the blood vessel, and are removed from the catheter unit 12 after the catheter unit 12 punctures the blood vessel.

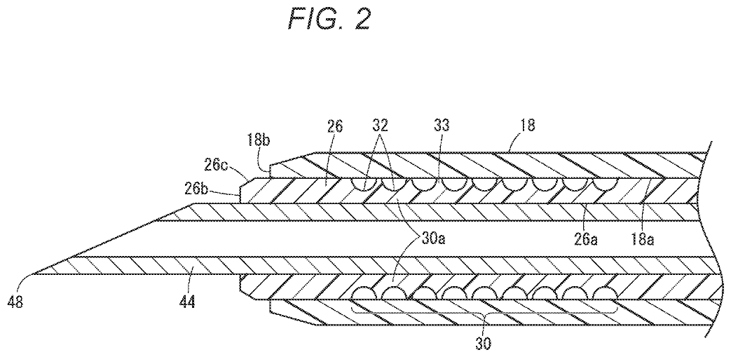

The catheter unit 12 includes an elongated tubular catheter 18 and a catheter hub 20 joined to a proximal portion of the catheter 18. The catheter 18 is a member indwelling in the blood vessel 104. As illustrated in FIG. 2, a first lumen 18a extending through the catheter 18 in an axial direction is formed inside the catheter 18. A distal portion 18b formed to be blunt (not sharp) is provided at a distal end of the catheter 18. The catheter 18 is made of a flexible resin material to prevent damage to the blood vessel 104.

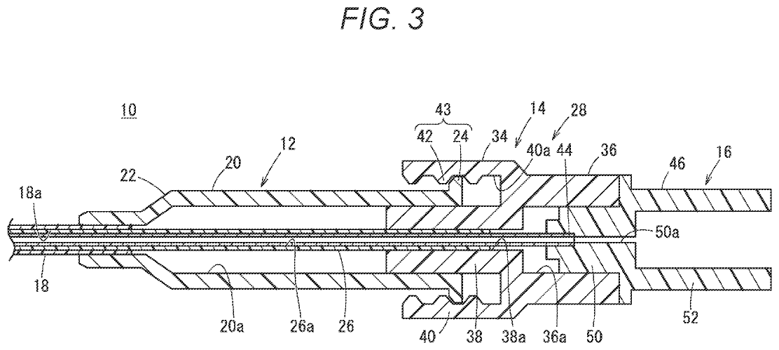

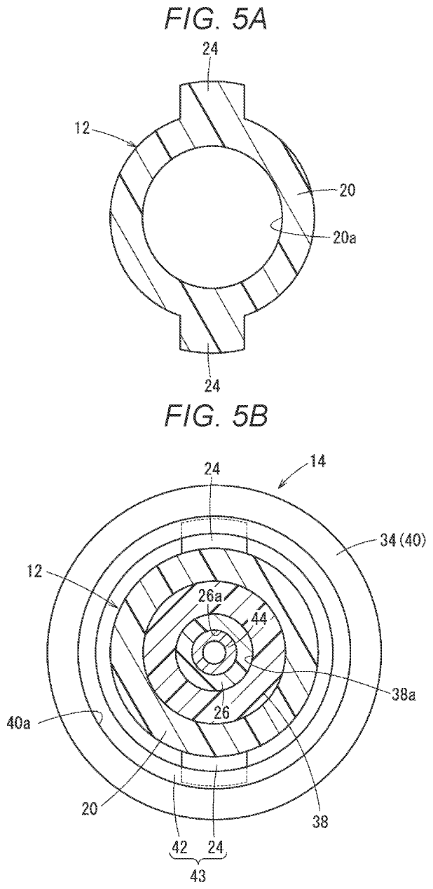

As illustrated in FIG. 3, the catheter hub 20 is formed in a tubular shape having an outer diameter larger than that of the catheter 18. A connection portion 22 whose diameter is reduced in a tapered shape toward a distal end thereof is formed at a distal end of the catheter hub 20, and the catheter 18 is liquid-tightly joined to the connection portion 22. A hollow portion 20a that is in communication with the first lumen 18a of the catheter 18 is formed inside the catheter hub 20. First projections 24 protruding outward in a flange shape are formed on a proximal end of the catheter hub 20. As illustrated in FIG. 5B, at least one pair of first projections 24 are formed at positions facing each other on at least a part of the catheter hub 20 in a circumferential direction. The catheter hub 20 is made of a resin material harder than that of the catheter 18.

As illustrated in FIG. 3, the reinforcement needle unit 14 includes a reinforcement needle 26 and a reinforcement needle hub 28 connected to a proximal end of the reinforcement needle 26. The reinforcement needle 26 is a hollow cylindrical member elongated in the axial direction. As illustrated in FIG. 2, a second lumen 26a that penetrates in the axial direction is formed inside the reinforcement needle 26. The reinforcement needle 26 has an outer diameter that makes the reinforcement needle 26 capable of being inserted into the first lumen 18a of the catheter 18, and is inserted into the catheter 18. The reinforcement needle 26 is formed longer than the catheter 18, and in an initial state, a distal portion 26b thereof slightly protrudes from the distal portion 18b of the catheter 18. The distal portion 26b of the reinforcement needle 26 is formed blunt to prevent damage to the blood vessel 104, and a chamfered portion 26c in which a sharp edge is removed is formed on an outer peripheral portion of the distal portion 26b.

The reinforcement needle 26 is harder and more rigid than the catheter 18 in order to reinforce the flexible catheter 18. Examples of materials of the reinforcement needle 26 include a metal material such as stainless steel and a hard resin material. Further, the reinforcement needle 26 includes a flexible portion formed in a vicinity of the distal portion 26b such that a vicinity of a distal end of the reinforcement needle 26 can be bent. The flexible portion 30 is formed as a tubular soft part having a predetermined length in the axial direction. The flexible portion 30 includes a thin portion 30a formed thinner than other portions of the reinforcement needle 26, and is formed more flexible than other portions of the reinforcement needle 26. The flexible portion 30 is provided closer to the proximal end than the distal portion 18b of the catheter 18, and is hidden inside the catheter 18. A length of the flexible portion 30 in the axial direction may be, for example, 3 mm to 10 mm.

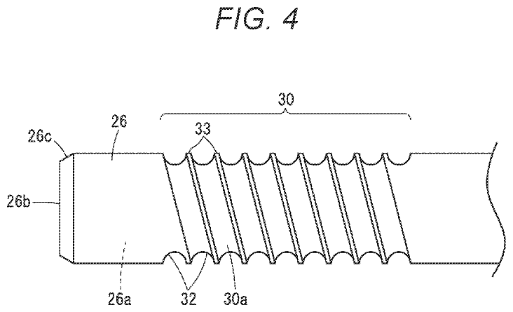

As illustrated in FIG. 4, the flexible portion 30 according to the present embodiment has a concavo-convex structure including a plurality of concave portions 32 and a plurality of convex portions 33 provided adjacent to the concave portions 32, and both are provided on an outer peripheral portion of the reinforcement needle 26. Portions formed thin due to the concave portions 32 give flexibility to the reinforcement needle 26. The concave portion 32 of the flexible portion 30 is not limited to a groove including a semicircular cross section as illustrated, and may be a rectangular groove. Further, the concave portion 32 may be formed as a notch having a narrower width. Further, a pattern of the plurality of concave portions 32 is not limited to a spiral shape, and may be formed in a mesh shape, a corrugated shape, or the like.

As illustrated in FIG. 3, the reinforcement needle hub 28 is provided on the proximal end of the reinforcement needle 26. The reinforcement needle hub 28 includes a catheter hub connection portion 34 provided on a distal end thereof and an inner needle hub mounting portion 36 provided on a proximal end thereof. The catheter hub connection portion 34 has a structure similar to a male luer lock, and includes a tip 38 that protrudes to the distal end side and is inserted into the catheter hub 20, and a tubular lock portion 40 that surrounds an outer periphery of the tip 38.

The tip 38 includes a shaft hole 38a that penetrates in the axial direction. The shaft hole 38a has an inner diameter capable of accommodating the reinforcement needle 26. A proximal portion of the reinforcement needle 26 is joined to the shaft hole 38a by a method such as adhesion. That is, the tip 38 holds the reinforcement needle 26. In the initial state, the tip 38 is inserted into the hollow portion 20a of the catheter hub 20, and the reinforcement needle hub 28 and the catheter hub 20 are liquid-tightly connected through the tip 38.

The lock portion 40 (a first fixing portion) is formed in a bottomed cylindrical shape surrounding an outside of the tip 38. On an inner peripheral surface 40a of the lock portion 40, a spiral second projection 42 that constitutes a screw structure to be screwed with the first projections 24 of the catheter hub is provided to protrude inward. The catheter hub 20 includes the first projections 24 on a proximal portion thereof. As illustrated in FIG. 5A, the first projections 24 of the catheter hub 20 are formed to protrude outward from at least a part of an outer peripheral portion of the catheter hub 20 in the circumferential direction. As illustrated, for example, a pair of the first projections 24 are provided at positions facing each other in the circumferential direction. A shape of the first projection 24 is not limited to the illustrated example, and one first projection 24 may be continuously formed over a range of at least a half circumference in the circumferential direction. Further, three or more first projections 24 may be provided separately in the circumferential direction.

As illustrated in FIG. 5B, the first projections 24 of the catheter hub 20 are screwed with the second projection 42 of the lock portion 40, and thus the catheter hub 20 and the reinforcement needle hub 28 are fixed such that the catheter hub 20 and the reinforcement needle hub 28 cannot be separated from each other by only a simple operation in a pull-out direction.

On the other hand, the inner needle hub mounting portion 36 on the proximal end of the reinforcement needle hub 28 is formed in a cylindrical shape having a circular hollow portion 36a therein, and the hollow portion 36a opens toward the proximal end. The hollow portion 36a is in communication with the shaft hole 38a.

As illustrated in FIG. 3, the inner needle unit 16 includes an inner needle 44 and an inner needle hub 46 connected to a proximal portion of the inner needle 44. The inner needle 44 is a hollow needle including a sharp needle tip 48 at a distal end thereof. An outer diameter of the inner needle 44 is smaller than an inner diameter of the second lumen 26a, and the inner needle 44 is inserted into the second lumen 26a of the reinforcement needle 26. The inner needle 44 is longer than the reinforcement needle 26, and in the initial state, the needle tip 48 of the inner needle 44 protrudes from the distal portion 26b of the reinforcement needle 26. The proximal portion of the inner needle 44 is held by the inner needle hub 46.

The inner needle hub 46 includes a fitting portion 50 (a second fixing portion) whose distal end is inserted into the inner needle hub mounting portion 36, and an inner needle hub main body portion 52 that extends to a proximal end of the fitting portion 50. The fitting portion 50 includes a shaft hole 50a and holds the inner needle 44 in the shaft hole 50a. The fitting portion 50 has an outer diameter that makes the fitting portion 50 capable of being inserted into the hollow portion 36a of the inner needle hub mounting portion 36. In the initial state, the fitting portion 50 is inserted into the hollow portion 36a of the inner needle hub mounting portion 36, and the inner needle hub 46 and the reinforcement needle hub 28 are connected to each other. The inner needle hub 46 and the reinforcement needle hub 28 are fixed to each other by a frictional force between the fitting portion 50 and an inner peripheral portion of the hollow portion 36a.

The inner needle hub main body portion 52 is a cylindrical member having an outer diameter that can be gripped, and is formed of a transparent or translucent resin material in order to facilitate visual observation of backflow of blood caused by flashback. The inner needle hub main body portion 52 and the fitting portion 50 are integrally formed.

In the present embodiment, a connection strength between the fitting portion 50 and the inner needle hub mounting portion 36 against a tensile force in the axial direction is smaller than a connection strength between the reinforcement needle hub 28 and the catheter hub 20 against a tensile force in the axial direction. Therefore, when the inner needle hub 46 is pulled, the inner needle unit 16 is pulled out earlier than the reinforcement needle unit 14.

The catheter assembly 10 according to the present embodiment is configured as described above, and operations thereof will be described below.

As illustrated in FIG. 6A, a user holds the catheter hub and the reinforcement needle hub 28 of the catheter assembly with one hand, and the needle tip 48 of the inner needle 44 punctures skin 102 of a patient toward the blood vessel 104. When the needle tip 48 of the inner needle 44 punctures the blood vessel 104, the blood inside the blood vessel 104 flows back through the inner needle 44 and flows into the inner needle hub 46. By visually observing the blood flashback into the inner needle hub 46, the user visually observes that the needle tip 48 has punctured the blood vessel 104.

Next, as illustrated in FIG. 6B, the user grips the inner needle hub main body portion 52 with the other hand and pulls out the inner needle hub main body portion 52 toward a direction of a proximal end of the catheter assembly 10. Thus, only the inner needle unit 16 is pulled out from the catheter assembly Because the second projection 42 of the reinforcement needle hub 28 is engaged with the first projections 24 of the catheter hub 20, the reinforcement needle unit 14 is not pulled out from the catheter assembly 10 by this operation.

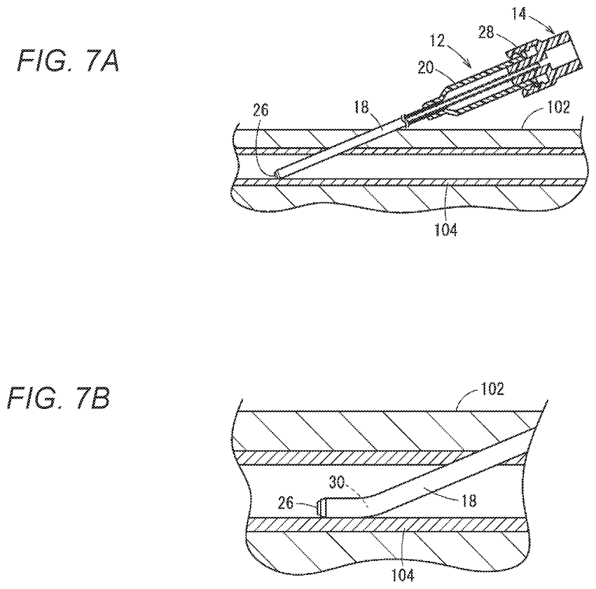

As illustrated in FIG. 7A, after the inner needle unit 16 is completely removed from the catheter assembly 10, the user pushes forward the catheter 18 into the blood vessel 104. In the present embodiment, the catheter 18 advances together with the reinforcement needle 26 in a state of being reinforced by the reinforcement needle 26 inside. Because deformation of the catheter 18 can be prevented by the reinforcement needle 26, an operation force from the catheter hub 20 and the reinforcement needle hub 28 is reliably transmitted to the distal end of the catheter 18. Therefore, the catheter 18 can be efficiently advanced in the blood vessel 104.

As illustrated in FIG. 7B, when the distal portions 18b and 26b of the catheter 18 and the reinforcement needle 26 reach a posterior wall of the blood vessel 104, the flexible portion of the reinforcement needle 26 is curved, and the catheter 18 is also curved accordingly. Therefore, it is possible to advance the catheter 18 into the blood vessel 104 while preventing an injury to the posterior wall of the blood vessel 104.

After the distal end of the catheter 18 is inserted into the blood vessel 104 by a desired length, the user rotates the reinforcement needle hub 28 with respect to the catheter hub 20 to release a locked state implemented by the second projection 42 of the reinforcement needle hub 28 and the first projections 24 of the catheter hub 20. Thereafter, the user pulls out the reinforcement needle hub 28 from the catheter hub 20, and completely removes the reinforcement needle unit 14 from the catheter unit 12. Thereafter, the user fixes the catheter hub on the skin 102 to complete indwelling of the catheter unit 12.

The catheter assembly 10 according to the present embodiment achieves the following effects.

The catheter assembly 10 according to the present embodiment includes: the catheter 18 having the first lumen 18a; the catheter hub 20 joined to a proximal end of the catheter 18; the reinforcement needle 26 inserted into the first lumen 18a of the catheter 18, having the second lumen 26a, and including the distal portion 26b formed blunt; the reinforcement needle hub 28 joined to the proximal end of the reinforcement needle 26; the inner needle 44 inserted into the second lumen 26a of the reinforcement needle 26 and including the sharp needle tip 48 at the distal end thereof; the inner needle hub 46 joined to a proximal end of the inner needle 44; the first fixing portion (for example, a latch section 43) that fixes the catheter hub 20 and the reinforcement needle hub 28 so as not to be detachable by an operation of pulling the catheter hub 20 and the reinforcement needle hub 28 in the axial direction; and the second fixing portion (for example, the fitting portion 50) that fixes the reinforcement needle hub 28 and the inner needle hub 46 to each other. The reinforcement needle 26 has higher rigidity than the catheter 18 and protrudes from the distal end of the catheter 18, and the inner needle 44 protrudes from the distal end of the reinforcement needle 26.

According to the above configuration, because the catheter 18 is reinforced by the reinforcement needle 26, when the catheter 18 is pushed forward into the blood vessel 104, the catheter 18 can be smoothly advanced even in a state in which the inner needle 44 is completely pulled out. Accordingly, the inner needle 44 can be completely pulled out after puncturing the blood vessel 104, and an event such as the needle tip 48 of the inner needle 44 damaging the posterior wall of the blood vessel 104 can be prevented.

In the catheter assembly 10, the fixation between the catheter hub 20 and the reinforcement needle hub 28 implemented by the first fixing portion may be stronger than the fixation between the reinforcement needle hub 28 and the inner needle hub 46 implemented by the second fixing portion. According to this configuration, when the inner needle hub 46 is pulled out, the reinforcement needle hub 28 can be prevented from being detached from the catheter hub 20, and the reinforcement needle 26 can be retained inside the catheter 18.

In the above catheter assembly 10, the first fixing portion may include the first projections 24 that protrude radially outward from the proximal end of the catheter hub 20, and the lock portion 40 that protrudes in a tubular shape from the reinforcement needle hub 28 toward the distal end of the reinforcement needle hub 28, surrounding at least a part of an outside of the catheter hub 20, and including the spiral second projection 42, that is screwed with the first projections 24, on the inner peripheral surface 40a facing the catheter hub 20. According to this configuration, the fixation between the catheter hub 20 and the reinforcement needle hub 28 implemented by the first fixing portion (the latch section 43) can be stronger than that implemented by the second fixing portion.

In the above catheter assembly 10, the first fixing portion may fix the catheter hub 20 and the reinforcement needle hub 28 to each other in the axial direction in a rotatable manner. In this case, a latch state implemented by the latch section 43 may be released by rotating the catheter hub 20 and the reinforcement needle hub 28 relative to each other.

In the above catheter assembly 10, the reinforcement needle 26 may include the flexible portion 30 enabling bending of the distal end of the reinforcement needle 26. According to this configuration, when the distal ends of the catheter 18 and the reinforcement needle 26 come into contact with the posterior wall of the blood vessel 104, the distal end of the catheter 18 can be flexibly curved, and the injury to the blood vessel 104 can be prevented.

In the above catheter assembly 10, the flexible portion may include a soft part provided at a position away from the proximal end further than away from the distal end. According to this configuration, the flexible portion 30 can be flexibly curved regardless of a rotational position of the reinforcement needle 26.

In the above catheter assembly 10, the flexible portion may have a concavo-convex structure including the plurality of concave portions 32 formed by thinning the outer periphery of the reinforcement needle 26 and the plurality of convex portions 33 provided adjacent to the concave portions 32.

### Second Embodiment

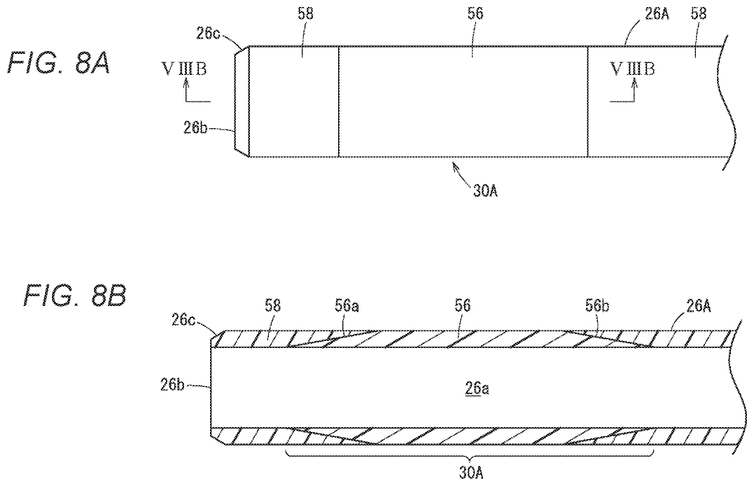

As illustrated in FIG. 8A, a reinforcement needle 26A according to the present embodiment is different from the reinforcement needle 26 in FIG. 4 in a flexible portion 30A. In the reinforcement needle 26A, the same configurations as those of the reinforcement needle 26 in FIG. 4 are denoted by the same reference numerals, and the detailed descriptions thereof are omitted.

In the reinforcement needle 26A, the flexible portion 30A includes an intermediate portion 56 formed of a material more flexible than those of other portions of the reinforcement needle 26A. The intermediate portion 56 is formed as a cylindrical member made of rubber, elastomer, or a resin material that is easily elastically deformed.

As illustrated in FIG. 8B, the intermediate portion 56 includes inclined surfaces 56a and 56b whose outer diameters gradually decrease on a distal end and a proximal end thereof, and is joined to a main body portion 58 of the reinforcement needle 26A via the inclined surfaces 56a and 56b. By joining via the inclined surfaces 56a and 56b, a joining area is increased, and the intermediate portion 56 can be more firmly joined to the main body portion 58.

As described above, in the reinforcement needle 26A according to the present embodiment, the flexible portion 30A is formed of a material more flexible than those of other portions of the reinforcement needle 26A. According to this configuration, a distal end of the reinforcement needle 26A can be flexibly curved, and the injury to the blood vessel 104 can be prevented.

### Third Embodiment

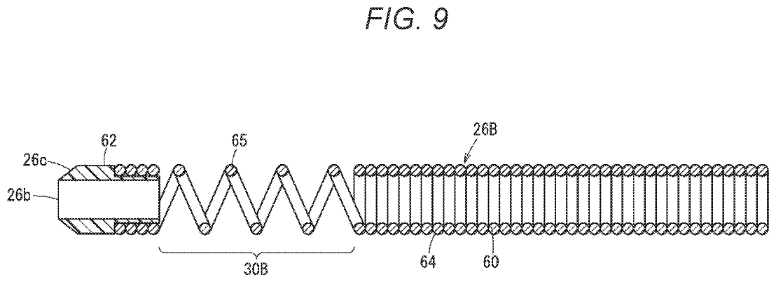

As illustrated in FIG. 9, a reinforcement needle 26B according to the present embodiment includes a wire 60 wound in a coil shape and a distal tip 62 connected to a distal end of the wire 60. The wire 60 includes a main body portion 64 densely wound in the axial direction and an intermediate portion sparsely wound. The intermediate portion 65 is provided near the distal end of the wire 60. The main body portion 64 is less likely to be curved because the wire 60 is densely wound, and the intermediate portion 65 is relatively easily to be curved and flexibly formed because the wire 60 is sparsely wound. In the reinforcement needle 26B according to the present embodiment, the intermediate portion 65 constitutes a flexible portion 30B.

The distal tip 62 is a tubular member including the distal portion 26b formed blunt, and the chamfered portion 26c is provided on an outer peripheral portion of the distal tip 62.

The above reinforcement needle 26B according to the present embodiment includes a rigid member (for example, the wire 60) that is wound along an outer peripheral portion of the reinforcement needle 26B from a proximal end to a distal end to impart rigidity, and the flexible portion 30B is formed by the rigid member wound at a sparse density. With this configuration, the distal end of the reinforcement needle 26B can also be flexibly curved, and the injury to the blood vessel 104 can also be prevented.

### Fourth Embodiment

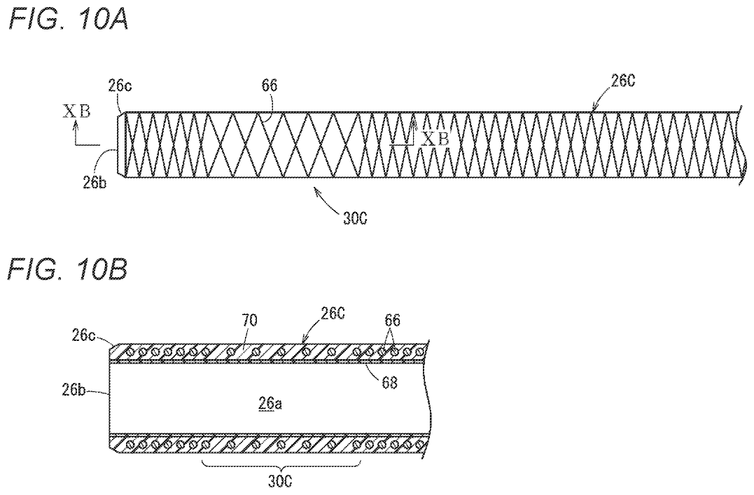

As illustrated in FIG. 10A, a reinforcement needle 26C according to the present embodiment has a structure in which a braided wire 66 is wound around an outer peripheral portion of the reinforcement needle 26C for reinforcement. In the reinforcement needle 26C, the same configurations as those of the reinforcement needle 26 in FIG. 4 are denoted by the same reference numerals, and the detailed descriptions thereof are omitted.

As illustrated in FIG. 10B, the reinforcement needle 26C includes an inner layer 68 formed in a thin tube shape. The inner layer 68 is flexibly formed. The braided wire 66 made of a metal wire is wound around an outer periphery of the inner layer 68 as a reinforcement structure. Further, the braided wire 66 is molded with an outer layer 70 made of resin.

A flexible portion 30C that can be flexibly curved is formed in a vicinity of the distal portion 26b of the reinforcement needle 26C. The flexible portion 30C is formed by winding the braided wire 66 more sparsely than that at other portions.

The reinforcement needle 26C according to the present embodiment also achieves the same effect as the reinforcement needle 26 according to the first embodiment.

### Fifth Embodiment

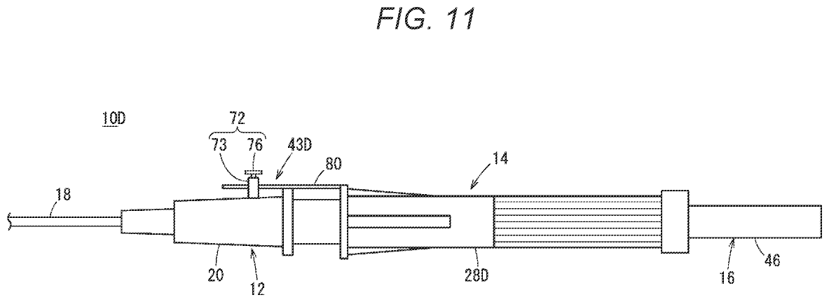

As illustrated in FIG. 11, a catheter assembly 10D according to the present embodiment is different from the catheter assembly 10 in FIG. 1 in a structure of a latch section 43D that engages the catheter hub 20 with a reinforcement needle hub 28D. In the catheter assembly 10D, the same configurations as those of the catheter assembly 10 are denoted by the same reference numerals, and the detailed descriptions thereof are omitted.

In the catheter assembly 10D, the latch section 43D (the first fixing portion) includes a lock mechanism portion 72 protruding from the outer peripheral portion of the catheter hub 20, and a lock pin 80 extending from the reinforcement needle hub 28D toward a distal end thereof.

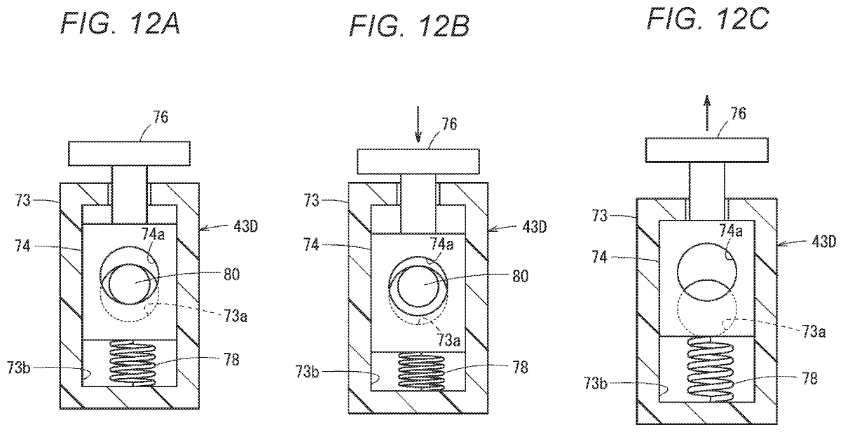

As illustrated in FIG. 12A, the lock mechanism portion 72 includes a housing 73 having a rectangular box shape when viewed from front. A first insertion hole 73a that penetrates in the axial direction of the catheter hub 20 is formed in the housing 73, and an accommodation chamber 73b is formed inside the housing 73. In the accommodation chamber 73b of the housing 73, a piston 74 that can slide in the accommodation chamber 73b in a vertical direction and a coil spring 78 that biases the piston 74 upward are accommodated.

A second insertion hole 74a that penetrates in the axial direction of the catheter hub 20 is formed in a center of the piston 74. Further, an operation portion 76 that receives an operation force from the user is provided on an upper portion of the piston 74. The first insertion hole 73a and the second insertion hole 74a each have inner diameters larger than a diameter of the lock pin 80.

In the initial state of the latch section 43D illustrated in FIG. 12A, the lock pin 80 is disposed such that the lock pin 80 passes through the first insertion hole 73a and the second insertion hole 74a. The piston 74 is biased upward by an elastic force of the coil spring 78, and the lock pin 80 is fixed in a pulling-out disabled manner in a state of being sandwiched between the first insertion hole 73a and the second insertion hole 74a. A fixation strength of the fixation between the catheter hub 20 and the reinforcement needle hub 28D implemented by the latch section 43D is greater than a fixation strength of a fixation between the inner needle hub 46 and the reinforcement needle hub 28D.

As illustrated in FIG. 12B, when the user presses the operation portion 76, the piston 74 is pushed down, and the first insertion hole 73a and the second insertion hole 74a overlap each other. As a result, the fixed lock pin 80 is released, and the reinforcement needle hub 28D can be pulled out from the catheter hub 20.

As illustrated in FIG. 12C, when the reinforcement needle hub 28D is pulled out from the catheter hub 20, the lock pin 80 is removed from the first insertion hole 73a and the second insertion hole 74a. When the user stops pressing the operation portion 76, the piston 74 is biased by the coil spring 78 and pushed up to an upper end of the accommodation chamber 73b.

As described above, according to the catheter assembly 10D according to the present embodiment, a fixed state implemented by the latch section 43D can be easily released by an operation of pushing down the operation portion 76. Because the release in the latch section 43D can be operated by pressing the operation portion 76 with one hand, operability is improved.

### Sixth Embodiment

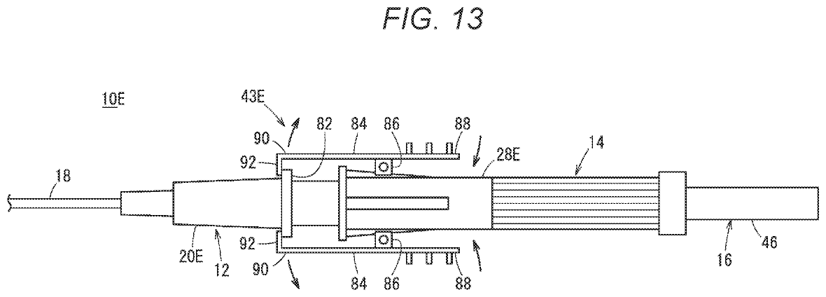

As illustrated in FIG. 13, a catheter assembly 10E according to the present embodiment is different from the catheter assembly 10 in FIG. 1 in a structure of a latch section 43E that engages a catheter hub 20E with a reinforcement needle hub 28E. In the catheter assembly 10E, the same configurations as those of the catheter assembly 10 are denoted by the same reference numerals, and the detailed descriptions thereof are omitted.

The catheter assembly 10E includes lock levers 84 that engage with a flange portion 82 at a proximal end of the catheter hub 20E. A pair of lock levers 84 are provided on a side portion of the reinforcement needle hub 28E. Each lock lever 84 is attached to the reinforcement needle hub 28E via a fulcrum 86 provided in a vicinity of a center of the lock lever 84. The lock lever 84 includes a grip portion 88 extending further toward a proximal end than the fulcrum 86 and an L-shaped arm portion 90 extending further toward a distal end than the fulcrum 86. A claw portion 92 that engages with a proximal end of the flange portion 82 of the catheter hub 20E is formed at a distal end of the arm portion 90.

The fulcrum 86 rotates the lock lever 84 about an axis perpendicular to a paper surface in FIG. 13, and incorporates a biasing member that biases the claw portion 92 in a direction approaching the catheter hub 20E. In the initial state, the claw portions 92 are engaged with the flange portion 82 of the catheter hub 20E to fix the catheter hub 20E and the reinforcement needle hub 28E.

When the grip portions 88 of the pair of lock levers 84 are gripped and operated to approach each other, the claw portions 92 are disengaged from the flange portion 82, and a fixed state between the catheter hub 20E and the reinforcement needle hub 28E is released.

As described above, according to the catheter assembly 10E according to the present embodiment, the fixed state implemented by the latch section 43E can be easily released by an operation on the lock levers 84. Because the lock levers 84 can be operated with one hand, the catheter assembly 10E is excellent in operability.

Although preferred embodiments of the invention have been described above, the invention is not limited to the above embodiments, and it is needless to say that various modifications can be made without departing from the gist of the invention.

What is claimed is:

1. A catheter assembly comprising:
a catheter having a first lumen;
a catheter hub joined to a proximal end of the catheter;
a reinforcement needle inserted through the first lumen of the catheter, having a second lumen, and including a distal portion that is blunt;
a reinforcement needle hub joined to a proximal end of the reinforcement needle;
an inner needle inserted through the second lumen of the reinforcement needle and including a sharp needle tip at a distal end thereof;
an inner needle hub joined to a proximal end of the inner needle;
a first fixing portion configured to fix the catheter hub and the reinforcement needle hub so as not to be detachable by an operation of pulling the catheter hub and the reinforcement needle hub in an axial direction; and
a second fixing portion configured to fix the reinforcement needle hub and the inner needle hub to each other; wherein:
a rigidity of the reinforcement needle is higher than a rigidity of the catheter, and the reinforcement needle protrudes from a distal end of the catheter; and
the inner needle protrudes from a distal end of the reinforcement needle.

2. The catheter assembly according to claim 1, wherein:
the fixation between the catheter hub and the reinforcement needle hub implemented by the first fixing portion is stronger than the fixation between the reinforcement needle hub and the inner needle hub implemented by the second fixing portion.

3. The catheter assembly according to claim 2, wherein:
the first fixing portion comprises:
a first projection that protrudes radially outward from a proximal end of the catheter hub, and
a lock portion that protrudes in a tubular shape from the reinforcement needle hub in a distal direction, surrounds at least a part of an outside of the catheter hub, and comprises a spiral second projection, which is screwed with the first projection, on an inner peripheral surface of the lock portion facing the catheter hub.

4. The catheter assembly according to claim 1, wherein:
the first fixing portion fixes the catheter hub and the reinforcement needle hub to each other in the axial direction in a rotatable manner.

5. The catheter assembly according to claim 2, wherein:
the first fixing portion fixes the catheter hub and the reinforcement needle hub to each other in the axial direction in a rotatable manner.

6. The catheter assembly according to claim 1, wherein:
the reinforcement needle comprises a flexible portion that enables bending of a distal end of the reinforcement needle.

7. The catheter assembly according to claim 2, wherein:
the reinforcement needle comprises a flexible portion that enables bending of a distal end of the reinforcement needle.

8. The catheter assembly according to claim 3, wherein:
the reinforcement needle comprises a flexible portion that enables bending of a distal end of the reinforcement needle.

9. The catheter assembly according to claim 6, wherein:
the flexible portion comprises a soft part provided at a position separate from the proximal end by a first distance, and separated from the distal end by a second distance that is less than the first distance.

10. The catheter assembly according to claim 7, wherein:
the flexible portion comprises a soft part provided at a position separate from the proximal end by a first distance, and separated from the distal end by a second distance that is greater than the first distance.

11. The catheter assembly according to claim 8, wherein:
the flexible portion comprises a soft part provided at a position separate from the proximal end by a first distance, and separated from the distal end by a second distance that is greater than the first distance.

12. The catheter assembly according to claim 9, wherein:
the flexible portion has a concave-convex structure comprising a plurality of concave portions formed by thinning an outer periphery of the reinforcement needle, and a plurality of convex portions provided adjacent to the concave portions.

13. The catheter assembly according to claim 10, wherein:
the flexible portion has a concave-convex structure comprising a plurality of concave portions formed by thinning an outer periphery of the reinforcement needle, and a plurality of convex portions provided adjacent to the concave portions.

14. The catheter assembly according to claim 9, wherein:
a material from which the flexible portion is formed is more flexible than a material from which other portions of the reinforcement needle.

15. The catheter assembly according to claim 10, wherein:
a material from which the flexible portion is formed is more flexible than a material from which other portions of the reinforcement needle.

16. The catheter assembly according to claim 11, wherein:
a material from which the flexible portion is formed is more flexible than a material from which other portions of the reinforcement needle.

17. The catheter assembly according to claim 6, wherein:
the reinforcement needle comprises a rigid member that is wound along an outer peripheral portion from the proximal end to the distal end to impart rigidity to the reinforcement needle, and the flexible portion is formed by a portion of the rigid member being wound at a lower density than a remained of the rigid member.

18. The catheter assembly according to claim 7, wherein:
the reinforcement needle comprises a rigid member that is wound along an outer peripheral portion from the proximal end to the distal end to impart rigidity to the reinforcement needle, and the flexible portion is formed by a portion of the rigid member being wound at a lower density than a remained of the rigid member.

19. The catheter assembly according to claim 8, wherein:
the reinforcement needle comprises a rigid member that is wound along an outer peripheral portion from the proximal end to the distal end to impart rigidity to the reinforcement needle, and the flexible portion is formed by a portion of the rigid member being wound at a lower density than a remained of the rigid member.

20. A method for inserting a catheter into a blood vessel of a patient, the method comprising:
providing a catheter assembly comprising:
the catheter, which has a first lumen,
a catheter hub joined to a proximal end of the catheter,
a reinforcement needle inserted through the first lumen of the catheter, having a second lumen, and including a distal portion that is blunt,
a reinforcement needle hub joined to a proximal end of the reinforcement needle,
an inner needle inserted through the second lumen of the reinforcement needle and including a sharp needle tip at a distal end thereof,
an inner needle hub joined to a proximal end of the inner needle,
a first fixing portion configured to fix the catheter hub and the reinforcement needle hub so as not to be detachable by an operation of pulling the catheter hub and the reinforcement needle hub in an axial direction, and
a second fixing portion configured to fix the reinforcement needle hub and the inner needle hub to each other, wherein:
a rigidity of the reinforcement needle is higher than a rigidity of the catheter, and the reinforcement needle protrudes from a distal end of the catheter, and
the inner needle protrudes from a distal end of the reinforcement needle;
holding the catheter hub 20 and the reinforcement needle hub of the catheter assembly;
puncturing the blood vessel of the patient with the needle tip of the inner needle such that blood inside the blood vessel flows back through the inner needle and flows into the inner needle hub;
gripping the inner needle hub and pulling the inner needle hub toward a direction of a proximal end of the catheter assembly, while the reinforcement needle and the reinforcement needle hub are not pulled out from the catheter assembly;
after the inner needle and the inner needle hub are completely removed from the catheter assembly, pushing forward the catheter into the blood vessel in a state of being reinforced by the reinforcement needle; and
after the distal end of the catheter is inserted into the blood vessel by a desired length, releasing a locked state between the catheter hub and the reinforcement needle hub, pulling out the reinforcement needle hub from the catheter hub 20 so as to completely remove the reinforcement needle and the reinforcement needle hub from the catheter and the catheter hub.
